(19) United States
(12) Patent Application Publication
(10) Pub. No.: US 2006/0246118 A1
(43) Pub. Date: Nov. 2, 2006

(54) Process of heating a carrier on which or inside which a cosmetic product is placed

(76) Inventor: Jean-Louis H. Gueret, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/541,840

(22) PCT Filed: Jan. 12, 2004
(86) PCT No.: PCT/EP04/00130

§ 371 Date: Jul. 10, 2006

Related U.S. Application Data
(60) Provisional application No. 60/443,556, filed on Jan. 30, 2003.

(30) Foreign Application Priority Data
Jan. 13, 2003 (FR) 03/00301

(51) Int. Cl. A61K 9/70

(52) U.S. Cl. 424/443

(57) ABSTRACT

The object of the present invention is a process for raising the temperature of a cosmetic product (1), in particular a skin care or hair care product, the product being placed on or in a carrier (2), for example impregnated on a carrier of this kind. The rise in temperature is obtained by an external heat source, for example by microwave radiation. The carriers concerned are for example wipes, patches, or any other material capable of holding the product and of being heated in a microwave oven.

The invention relates to make-up processes and processes for application of a skin care product, and also to devices used in the implementation of such processes.

It has been proposed to apply cosmetic products after having raised their temperature.

Thus, French patent application FR 2 376 401 proposes that shampoos be heated to a temperature slightly higher than that of the human body in order to achieve improved effectiveness. To this end, quantities of shampoo are placed in an apparatus comprising heating elements.

U.S. Pat. No. 5,856,653 describes a device intended to heat receptacles containing mascara so as to liquefy the latter.

U.S. Pat. No. 5,775,344 describes a packaging and applicator device for mascara comprising a heating element integral with the container.

International application WO 00/43286 describes a device comprising components which, when mixed, produce an exothermic reaction enabling the temperature of a cosmetic composition to be raised.

U.S. Pat. No. 4,913,957 discloses a laminated carrier comprising a first layer forming a closed pocket capable of storing heat in a manner designed to diffuse the heat towards a second adjacent layer of the carrier, this second layer being saturated with product and intended to be applied against a surface to be treated.

The object of the invention, according to a first aspect inter alia, is a process for the application of a cosmetic product, in particular for skin care or hair care, the product being arranged on or in a carrier capable of being applied to a surface to be treated, the carrier being substantially dry to the touch, the process comprising the following steps:
applying a means of heating external to the carrier, in particular by microwave radiation, so as to raise the temperature at least of the product, and
applying the product to said surface.

In the invention, the term “substantially dry to the touch” preferably refers to a carrier containing less than 15% water relative to the weight of the carrier, and preferably less than 10% water, and more preferably less than 5% water.

Another object of the invention is a process for the application of a cosmetic product, in particular for skin care or hair care, this product being arranged on or in a carrier capable of being applied to a surface to be treated, the carrier comprising two respectively non-occlusive application surfaces, the process comprising the following steps:
applying a means of heating external to the carrier, in particular by microwave radiation, so as to raise the temperature at least of the product, and
applying the product to said surface.

Heating of the product presented in a form impregnated in a carrier can for example promote spreading of the product during application, its retention on the area treated, for example the eyelashes, the skin or the mucous membranes, or can produce at least two types of effects in relation to the temperature to which it is heated and used. The impregnated structure is preferably substantially flat and includes at least one portion capable of holding the product.

The purpose of the heating stage according to the invention is to facilitate liquefaction of the product substantially dry to the touch, so that it flows as required through the carrier, and so as to promote spreading of the product. In fact, application of the dry product is less pleasant than application of the same product in liquefied form. The process according to the invention has the advantage of proposing substantially dry carriers that are easy to handle, do not cause soiling when not in use, and in which the active agents of the product are protected during storage. The active agents in the product are released only after the heating stage which is immediately followed by the stage of applying the product to the surface to be treated.

Another object achieved by the heating stage according to the invention is to facilitate activation of at least one active component present in the product. For example activation corresponds to a physico-chemical modification of the product. physico-chemical modification is understood to mean any change affecting at least one chemical and/or physical characteristic of the product, and preferably of one of the possible active components of the product. This modification may involve several components of the product, and can promote catalysis of these components for example.

Secondarily, the heating stage can also have an impact on the physical and/or chemical characteristics of the carrier, and bring about their modification to improve the usage made of the carrier, in particular when it is applied against a surface to facilitate application of the product. In particular, the temperature of the carrier can also be raised intrinsically by this heating stage.

The rheological properties of the product can be modified by temperature, in particular viscosity, surface tension, structural composition, and thixotropic properties, where appropriate. Heating can thus facilitate take-up of the product. In effect, more product is released by the carrier the greater the tendency of the heat to fluidise the product and allow increased flow at the external surface of the carrier, which makes the product easier to apply. The bonds between the molecules and the various components of the product are modified, in particular any solid phase fats and waxes that it may include become liquid under the effect of this heat. They then draw the active components with them to the external surface.

Where the carrier includes two respectively non-occlusive application surfaces, these faces are opposite each other. The heating stage tends to fluidise the product and promote increased flow at the external surfaces respectively presented by each of these non-occlusive application surfaces. Generally only one of these two surfaces can be applied at once, and two effective application surfaces are therefore available enabling the same carrier to be used twice. Furthermore, certain carriers are faster to make in that the product penetrates more quickly by capillary action into said carrier. The term non-occlusive surface is understood to mean a surface capable of allowing at least part of the product held by the carrier to pass through.

Advantageously, the product possesses properties that enable it to be applied either hot or cold, in particular at ambient temperature. This can enable the user to adapt the properties of a product as effectively as possible to the type of application or make-up desired, in particular to apply the product hot when it is wished to spread it in a thinner layer to obtain a finer application of make-up, whereas this same product results in a thicker application and less uniform finish when applied cold. Heating therefore makes it possible to initiate and/or accelerate the appearance of a functional property corresponding to a physical or chemical characteristic of an active component of the product.

Furthermore, in relation to its chemical composition, the product can include an active component that is only capable of activation after heating to a certain temperature. In this case, it is possible for example to have a first cosmetic or skincare effect at a first temperature, for example at ambient temperature, and to obtain a second different cosmetic or skincare effect at a second temperature, for example higher than the first.

Hot application of the product can promote the penetration of an active constituent of the product at least into the skin, the mucous membranes or keratin fibres, and exert a local action on the blood circulation. In fact, in the case of topical application, the heat of the carrier is transmitted to the cells of the epidermis and promotes opening of the pores. The time required to apply the product is thus considerably reduced when it is presented in impregnated form in a carrier heated according to the invention.

In the case of reusable carriers, the product preferably includes thermo-reversible compounds to enable it to be re-used in the hot or cold state after the first use. The process can be put into effect once or several times, depending on whether or not the device is designed for single use.

Furthermore, when the product fluidises under the effect of the heat, the latter spreads through the structure of the carrier and softens its texture. The carrier becomes more pleasant to the touch and permits application to sensitive areas of the skin.

Also, any need to wet the carrier prior to use can be avoided by the fact that the active constituents of the product emerge spontaneously from the carrier structure during the heating stage. They are exuded at the surface of the carrier and can thus be directly applied to the skin.

Nevertheless, external heating of the carrier is in no way incompatible with a preliminary stage during which the carrier is immersed in a liquid, for example water, to increase the quantity of liquid impregnated in the carrier. Thus the heating operation, in particular by microwaves, facilitates initial heating of the liquid impregnated within the carrier and its overall fluidisation to facilitate its release from the carrier. The active components, whether hydrophilic or not, are all entrained by the liquid to the surface of the carrier.

These carriers are capable of causing active constituents to penetrate into the skin by transdermal action. Carriers capable of being impregnated with at least one active product for the purpose of application on a surface to be treated are for example selected from among wipes, patches, and generally all types of porous substrates. Preferably, these carriers have an oblong structure, i.e. of lesser thickness than the dimensions of the plane in which they are defined.

Another impregnated carrier can for example be a flock applicator, this applicator comprising for example an elastomer or plastics material body with a surface covering of a flock material impregnated with the product.

These carriers can have a dry or wet appearance depending on the agents included in them and the method of activation of these agents. Dry carriers can be used directly without wetting prior to use, provided that the active constituents of the product are fluidised and expelled from the carrier during the heating stage. Dry carriers can also be wetted or re-wetted before this heating stage.

The material constituting the carrier can be hydrophilic or hydrophobic depending on the modes of use and the active components impregnated in the carrier.

Cosmetic wipes are generally composed of a substrate made of a natural or synthetic material that is preferably non-woven, but which can also be a foam or a woven material, or a cellulose or silicone matrix. Said substrate is impregnated with a composition suitable for the intended purpose, for example cleansing or removal of make-up from the skin, or for skin care purposes. Wipes are customarily made in a material selected from among the following: non-woven material, in particular a ventilated or perforated non-woven material, a foam, a woven fabric, a felt material, a perforated or non-perforated plastic film, preferably without metallic coating.

Patches generally have a structure comprising several successive layers in the following order: a first layer, referred to as the carrier layer and generally occlusive, i.e. composed of a material that is impermeable to the active component so as to prevent evaporation of the latter and to facilitate transdermal penetration; a second layer, referred to as the reservoir layer, attached to the carrier layer and containing the product and therefore the active component, this reservoir layer being capable of coming into direct contact with the skin, and, if required, to facilitate attachment of the patch to the skin, a layer of an adhesive material applied to the surface of the reservoir layer and permeable to the active component; finally, a detachable protection layer covering and sealing the reservoir layer so as to protect it from any external contamination during storage prior to utilisation of the patch.

In a simplified manner, carriers are also known that are made for example of a woven or non-woven material and comprising a layer of product affixed to at least one of the surfaces of the carrier.

So-called “sandwich” masks are also known that have to be wetted before use and comprise an adhesive matrix arranged between at least two layers bonded in a permanent manner to the matrix; at least one of these layers and generally both layers are permeable to a liquid, and the matrix includes at least one principle active constituent capable of exerting its action on the skin when the matrix is placed in contact with said liquid. Preferably, the matrix contains one or more components that are soluble in said liquid; the liquid can be water or a water-alcohol solution. For example, the matrix includes microspheres of wax or solidified oil.

Preferably, such masks are obtained without passing through a prior aqueous phase for the impregnation of active constituents. Consequently, masks of this kind need not include preservatives in the product formulation. The quantity of product capable of being absorbed by the carrier therefore includes more active constituents. The carrier is for example impregnated directly with dehydrated or lyophilised product.

In the case of carriers that require wetting before use, cooperation between the matrix and the liquid facilitates the release of active constituents from the matrix, as the matrix includes components capable of expanding on contact with said liquid and therefore of increasing the mesh size of the carrier. For example moisture-absorbing compounds are able to expand, in particular those selected from the following list: polyacrylates, silica, cotton fibres, starches, alginates, calcium or magnesium carbonates, viscose, cellulose, lyophilisates.

Irrespective of whether or not the carrier requires wetting before use, the process according to the invention involving heating the carrier before use facilitates a high rate of entrainment of the product contained in the matrix or reservoir layer to at least one external surface of a permeable layer attached to said matrix, or to at least one external surface of said reservoir layer. The finer the permeable layer attached to the matrix, the greater the entrainment of product.

The carrier can be cut so as to take the form of a disc, a mask, a towel, a glove, a pre-formed roller, or any other shape suitable for cosmetic use.

The carrier is heated by means of an energy source external to the carrier. Heating can be achieved by placing the carrier in an oven, for example a microwave oven, or by placing it above a steam bath, under or on a heating element. In the case of carriers that require moistening before use, heating above a steam bath can provide a means of both heating and moistening the carrier at the same time. The heat source can be infrared, visible light, ultraviolet, electric, electromagnetic, or any other form capable of heating solid impregnated carriers.

In the invention, the carrier is preferably heated in a microwave oven. The term “microwave oven” means conventional ovens as used in other applications to heat food and including a chamber in which an item to be heated can be exposed to electromagnetic energy. In this case, the carrier of choice is advantageously devoid of metal, for example metal parts or metallic or electrically conductive coatings, and is preferably made only of materials compatible with use in a microwave oven. This method of heating allows a rapid rate of temperature rise to be achieved even if the carrier impregnated with product is made of one or more materials that are poor conductors of heat. The rise in temperature of the product within the mass of the carrier is rapidly achieved.

Heating is applied in relation to the melting point and/or liquefaction point of the active constituents to be recovered from the product impregnated in the carrier. The product can be heated so that its temperature is between 25° C. and 100° C., preferably between 30° C. and 55° C., for direct application onto the skin. In the case of heating by microwave radiation, the heating time can for example be between 1 and 150 seconds, preferably between 2 and 60 seconds, or between 3 and 25 seconds, being for example close to 5 seconds, depending on the microwave power selected.

Generally, the heating time depends principally on the power of the selected heat source, the nature of the carrier, the initial temperature and the temperature to be reached, and also on the quantity and nature of the product impregnated in the carrier.

Preferably, the carrier is laid “flat” in the heating means so that liquefaction of the product causes maximum impregnation of at least one of the surfaces of the carrier intended for application of the product. In effect, the fluidised product is directed by gravity or by virtue of the mesh structure of the carrier in a manner such that it spreads by capillary action and gravity to one of the external surfaces.

The carrier can be presented in a packaging device comprising a notice informing the user of the necessary heating time depending on the power of the oven. The packaging device can include a sign informing the user that it can be heated, and in particular that it can be placed in a microwave oven.

A further object of the invention, according to another of its aspects, taken independently or in combination with the foregoing, is a carrier on which or inside which is placed a cosmetic product, in particular for skin care or hair care, comprising an indicator designed to provide information relating to the temperature of the product. An indicator of this kind can inform the user about the temperature of the product when it is removed from the heating means, to enable the product to be used properly and to safeguard the user against being burned.

The indicator used can in particular serve to warn the user when the temperature of the product is higher than at least one pre-defined value, for example when the temperature of the product is between two pre-defined values corresponding to the temperature range enabling effective use of the product in the hot condition. The indicator can for example change state at a temperature to which it is desired to heat the product. If it is desired for example to use the product at a temperature T1, an indicator is used that changes colour at a temperature T2 several ° C. below T1, the temperature T2 being chosen in relation to the thermal resistance between the indicator and the product. This type of indicator can serve to warn the user.

The temperature sensitive indicator can for example change appearance, in particular its colour, with temperature, by changing from one colour to another when a pre-defined transition temperature is reached. The indicator can also present a colour saturation effect which is a function of temperature. The indicator can also present a transparency effect which is temperature dependent, becoming transparent or opaque when a certain temperature is reached. Preferably, the indicator changes its appearance in a reversible manner with temperature, i.e. it reverts to its initial appearance when the device returns to ambient temperature.

The indicator can for example include at least one flexible support fixed, for example by bonding, to the carrier. The indicator can for example take the form of a pad or an adhesive label affixed to a face of the carrier. The indicator can also be made by printing on the carrier, for example using an ink comprising a thermochromic pigment. The indicator can also be formed by incorporating a thermochromic pigment into the material of the carrier or the product.

The indicator can include any material which changes its appearance with temperature, for example cholesteric liquid crystals, possibly encapsulated, and preferably a material compatible with placement of the device in a microwave oven. Examples of materials which change their appearance with temperature are described for example in patent application EP 1 191 317 A1 or in U.S. Pat. No. 5,786,578.

A further object of the invention is a carrier on which or inside which is placed a cosmetic product, in particular for skin care or hair care, said carrier comprising two respectively non-occlusive application surfaces, the carrier being capable of heating in a microwave oven and comprising on at least part of its surface means capable of changing appearance, in particular colour, in response to an increase in temperature.

Another object of the invention, taken independently or in combination with the foregoing, is a method for promoting the sale of a carrier impregnated with a cosmetic product, including a skin care product, which makes known the possibility of placing the device in a microwave oven to raise the temperature of the product in order for example to modify the properties of the latter, in particular its rheology. This product is for example a skin care or make-up product, such as a make-up removal wipe or a two-layer mask.

A further object of the invention, taken independently or in combination with the foregoing, is a method for promoting the sale of a carrier impregnated with a cosmetic product, which makes known the possibility of obtaining two different make-up effects according to whether the product is used hot or cold.

Promotion of the product can be effected through any channel of communication. This may be done by a vendor, directly at the point of sale (through a beauty establishment or hairdressing salon for example), or through a sales channel, in particular mail order, and via radio or television, in particular in the context of advertising commercials. It may also done through press publications or by means of any other document, in particular for advertising purposes. It may also be done through a computer network or mobile telephone network. It may also be done on the packaging device, other packaging media or in accompanying instructions.

The invention will be better understood by reading the following description and by reference to the accompanying drawings. These are given for guidance only and in no way limit the scope of the invention. The figures show:

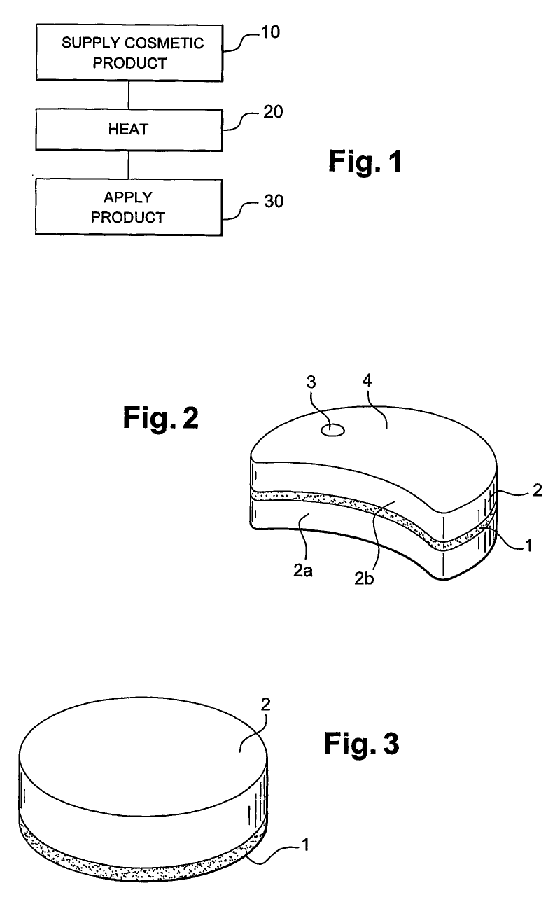

FIG. 1 is a block diagram showing the stages of an example of the process according to the invention,

FIGS. 2 and 3 are profile views of impregnated carriers to be heated according to the invention;

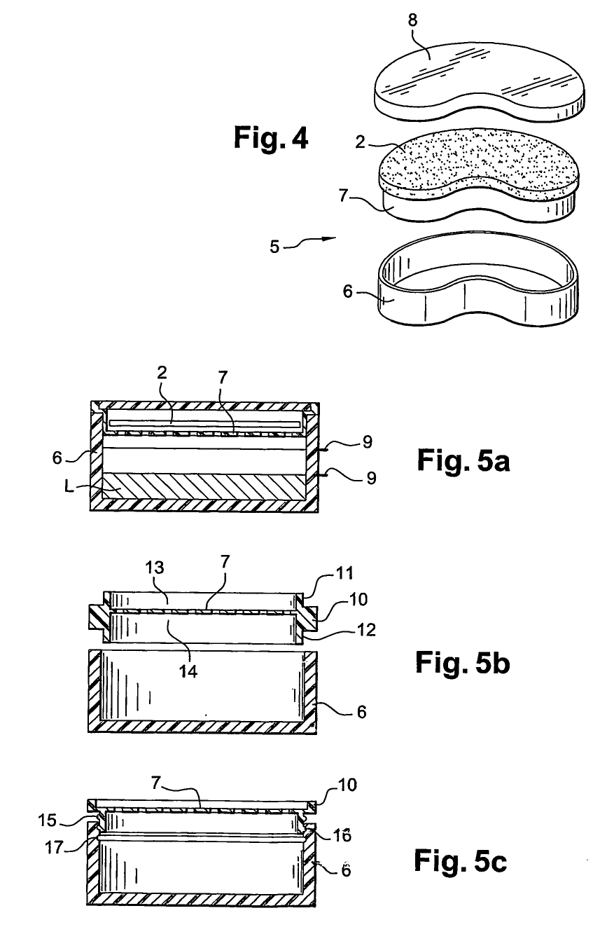

FIG. 4 is an exploded view of a device to enable heating above a steam bath,

FIGS. 5a), 5b), and 5c) are examples of embodiments of the device according to FIG. 4.

The block diagram in FIG. 1 illustrates different stages in a process which can be used to raise the temperature of a cosmetic product 1, FIG. 2, impregnated in a carrier 2, according to a first aspect of the invention.

This process includes a first stage 10 involving impregnation of the cosmetic product in a carrier. Impregnation of the cosmetic product on the carrier can be accomplished according to known techniques, in particular those used to make patches or wipes.

The process then includes a stage 20 which involves heating the product by a heating means external to the carrier and the product. For example, this heating means is a microwave oven in which case the carrier does not include any metallic or electrically conductive parts liable to be damaged on exposure to microwave radiation or to damage the oven used.

The effectiveness of microwave heating is related to the fact the power output of the oven is very easy to adjust, and furthermore the heating time can also be closely monitored. Thus, it is possible to give indicative heat setting combinations for a given carrier, for example of the type (P1, t1); (P2, t2) etc where ‘P’ denotes the power of the oven, and ‘t’ the heating time.

Preferably, a microwave power setting is selected so that the time for which the product is exposed to microwave radiation is relatively short, for example less than 20 seconds, in particular of the order of several seconds.

The packaging device is then taken out of the heating means and step 30 is carried out, involving application of the product. This application can be accomplished for example by applying the carrier directly against a surface to be treated, for example the skin. The product can for example be a make-up remover, a moisturiser, a foundation, a sunscreen product, a hair product, a self-tanning product, or any other skin care product, without this list being limitative.

At the end of the heating time in the microwave oven, the temperature of the product at the external surface of the carrier can exceed 55° C., or 70° C. For this reason, in a preferred embodiment, the carrier comprises a temperature indicator 3.

This temperature indicator 3 can take the form for example of a label or pad bonded to an application surface of the carrier, as illustrated in FIG. 2. The temperature indicator can also be made by mixing into the material intended for example to form the product layer 1 a known thermochromic pigment or any other material that changes colour with temperature and preferably is compatible with placement of the carrier in a microwave oven to heat the product. It is also possible to screen print a stripe or any other pattern using an ink comprising a thermochromic pigment onto an external surface 4 of the carrier 2.

According to another aspect of the invention, it is possible to select the carrier material such that it possesses a sufficiently high thermal inertia that the product impregnated therein does not cool down too quickly. The carrier is then made of a plastics material comprising a large proportion of a mineral or other charge imparting to it an elevated thermal capacity.

It is thus possible for example to produce a moulded thermoplastic or thermo-hardening material comprising a charge of a compound such as bronze or an aluminium oxide. It is in particular possible to make the carrier by moulding a mixture comprising 60% by weight of aluminium oxide and the remainder of polyamide or polypropylene. By way of a further non-limitative example, the carrier can also be made by moulding a mixture comprising 40% by weight of polypropylene and 60% of a ceramic material.

The fact that the thermal inertia of the carrier is relatively large, by virtue of the use of a charged plastics material for example, can make it possible to reduce the electric power consumption of the heating means.

The thermal inertia of the carrier can also be increased by using a material to make the carrier that is capable of becoming internally loaded with the product to be applied, for example a porous material such as a foam or a frit. The product contained in the pores of the carrier can be heated when placed in the microwave oven and thermal inertia can be conferred to the carrier.

The carrier can also include a symbol indicating the possibility of placing the carrier in a microwave oven.

FIG. 2 illustrates a carrier of the “two-layer” type, in that it includes a product layer 1 surrounded on both sides respectively by a first and second outer layer, respectively 2a) and 2b). At least one of these outer layers is made of a material that can be traversed by the product and capable of being applied against a surface to be treated. In the present case, the outer layers are shaped so as to present a plane surface, a limited thickness and an outline adapted to its intended area of application. For example, the product layer conforms to the same surface area and after heating can coat and pass through the entire surface area of the at least one permeable layer.

After heating, the active constituents are fluidised and diffuse through the permeable layer.

In a variant according to FIG. 3, the carrier includes a single layer on which a product layer 1 is directly presented. The action of heating this type of carrier is intended to induce a change in at least one physico-chemical characteristic of the product when this product is to be directly applied to an area to be treated.

In cases where it is desired to moisten the carrier while heating, according to the invention, the carrier to be heated is mounted on a steaming device which is then placed in a microwave oven. This steaming device 5 includes a container 6 in which a quantity of a vaporisable liquid L, for example water, can be stored. A grid 7 is mounted above this container 6, allowing the steam evaporating from the container to pass through the grid to moisten a carrier 2 arranged on the grid 7. Also, optionally, this device 5 can include a lid 8 to ensure continuous vaporisation of the liquid, and as the steam comes into contact with the lid it condenses to liquid again and flows back into the container.

The height of the liquid level in the container 6 and the positioning of the grid 7 relative to this liquid level can be adjusted to obtain a greater or lesser degree of moistening. With regard to the liquid level, the container 6 has graduations 9 for example, enabling the quantity of water placed in the container to be measured with a minimum of precision. According to FIG. 5a, the container 6 includes such graduations 9.

The grid 7 or the container 6 also have fixing means capable of engaging together to facilitate mounting respectively one on the other. According to FIG. 5b, the grid 7 is mounted inside a hollow arrangement 10 of which the outside rim is adapted to fit inside the container 6. For example, if the container 6 is cylindrical, the grid 7 is mounted inside a toroidal structure. An outer periphery of this toroidal structure 10 comprises a first indentation 11 on the side of a first face 13, for example the upper face 13 of the grid 7. Furthermore, the structure 10 has a second indentation 12 on the side of a second face 14 opposite the first face 13 of the same grid 7. As the grid 7 is preferably not positioned centrally between these two indentation levels 11 and 12, depending on which indentation in placed in cooperation with the inner rim of the container 6, the grid 7 is arranged at two different levels relative to the container 6.

Reciprocally, the indentations 11 and 12 can be formed on an inner edge of the structure 10 to engage with an outer rim of the container 6.

In a variant according to FIG. 5c, the grid 7 is mounted inside a structure 10 comprising on its outer rim at least two flanges 15 and 16 intended to engage respectively with a setback 17 provided in an inner rim of the container 6. The structure 10 is slightly flexible in order to accommodate a radial stress allowing the structure 10 to be pushed onto the container 6. Thus, when the first flange 16 is inserted into the setback 17, the second flange 15 is preferably not under strain. On the other hand, when the carrier 10 is pushed further into the container 6, the first flange 16 is then held under strain against the inner rim while the second flange 15 engages with the setback 17.

Throughout the description, including the claims, the expression “including a” should be understood to be synonymous with “including at least one”, unless otherwise specified.

1-17. (canceled)

18. A method of applying a cosmetic product to a surface, the method comprising:
increasing temperature of a cosmetic product arranged on or in a carrier substantially dry to the touch, the carrier being capable of being applied to a surface, and the temperature of the cosmetic product being increased via an energy source external to the carrier; and
applying the product to the surface.

19. The method of claim 18, wherein the product comprises a skin care product or a hair care product.

20. The method of claim 18, wherein the increasing temperature comprises increasing the temperature of the cosmetic product via microwave radiation.

21. The method of claim 18, wherein the increase in the temperature of the cosmetic product enables activation of at least one constituent of the cosmetic product.

22. The method of claim 18, wherein the increase in the temperature of the cosmetic product initiates or promotes activity of at least one constituent of the cosmetic product.

23. The method of claim 22, wherein the increase in the temperature of the cosmetic product promotes release of the at least one constituent to the surface.

24. The method of claim 18, wherein the increase in the temperature of the cosmetic product enables liquefaction of the product.

25. The method of claim 18, wherein the carrier is in the form of at least one layer.

26. The method of claim 18, wherein the carrier is made of a material comprising at least one of: cellulose, foam, woven material, felt, non-woven material, and plastic material.

27. The method of claim 18, wherein the carrier is immersed in a liquid before being heated.

28. The method of claim 27, wherein the liquid comprises water.

29. The method of claim 18, wherein the increasing comprises increasing the temperature of the cosmetic product to a temperature between 25° C. and 100° C.

30. The method of claim 29, wherein the increasing comprises increasing the temperature of the cosmetic product to a temperature between 30° C. and 55° C.

31. The method of claim 20, wherein the cosmetic product is exposed to the microwave radiation for a time period between 1 second and 150 seconds.

32. The method of claim 31, wherein the cosmetic product is exposed to the microwave radiation for a time period between 2 seconds and 60 seconds.

33. The method of claim 32, wherein the cosmetic product is exposed to the microwave radiation for a time period between 3 seconds and 25 seconds.

34. The method of claim 18, wherein the carrier is on a grid above or inside the container, wherein the container contains a liquid vaporizable at a heating temperature.

35. The method of claim 34, wherein the height of the grid is adjustable relative to the level of liquid in the container.

36. The method of claim 34, further comprising a lid arranged above the grid, wherein the lid and the container define a substantially closed volume around the carrier.

37. The method of claim 18, wherein the carrier includes a temperature indicator.

38. The method of claim 37, wherein the indicator is configured to change its appearance as a function of the temperature.

39. The method of claim 38, wherein the indicator is configured to change its color as a function of the temperature.

40. The method of claim 37, wherein the indicator is configured to change its material state at a predetermined temperature.

41. The method of claim 37, wherein the indicator comprises an ink or a label comprising a thermochromic pigment.

42. The method of claim 37, wherein the indicator is placed on at least part of a surface of the carrier.

43. The method of claim 41, wherein the indicator is placed on at least part of the surface of the carrier by printing.

44. A method of applying a cosmetic product to a surface, the method comprising:
increasing temperature of a cosmetic product arranged on or in a carrier comprising two non-occlusive application surfaces, the carrier being capable of being applied to a surface and the temperature of the cosmetic product being increased via an energy source external to the carrier; and
applying the product to the surface.

45. The method of claim 44, wherein the cosmetic product comprises a skin care product or a hair care product.

46. The method of claim 44, wherein the increasing temperature comprises increasing the temperature of the cosmetic product via microwave radiation.

47. The method of claim 44, wherein the increase in the temperature of the product facilitates liquefaction of the cosmetic product.

48. A carrier associated with a cosmetic product, the carrier comprising:
a body comprising a surface,
wherein at least part of the surface of the body comprises an indicator configured to change its appearance in response to an increase in temperature,
wherein the carrier is substantially dry to the touch, and
wherein the carrier is capable of being heated in a microwave oven.

49. The carrier of claim 48, wherein the cosmetic product is on or inside the carrier.

50. The carrier of claim 49, wherein the cosmetic product comprises a skin care product or a hair care product.

51. The carrier of claim 49, wherein the indicator is configured to change its color in response to a temperature increase.

52. A carrier associated with a cosmetic product, the carrier comprising:
a body comprising
a first non-occlusive application surface, and
a second non-occlusive application surface,
wherein at least part of one of the first and second surfaces comprises an indicator configured to change its appearance in response to an increase in temperature,
wherein the carrier is substantially dry to the touch, and
wherein the carrier is capable of being heated in a microwave oven.

53. The carrier of claim 52, wherein the cosmetic product is on or inside the carrier.

54. The carrier of claim 53, wherein the product is a skin care product or a hair care product.
